(19) United States
(12) Patent Application Publication
(10) Pub. No.: US 2026/0062380 A1
(43) Pub. Date: Mar. 5, 2026

(54) METHOD OF PREPARING BENZENESULFONYL COMPOUND

(71) Applicant: SK INNOVATION CO., LTD., Seoul (KR)

(72) Inventors: Seong Ho JIN, Daejeon (KR); Myoung Lae KIM, Daejeon (KR)

(21) Appl. No.: 19/304,602

(22) Filed: Aug. 20, 2025

(30) Foreign Application Priority Data
Sep. 4, 2024 (KR) 10-2024-0119927

(51) Int. Cl. C07C 303/26

(52) U.S. Cl. CPC C07C 303/26

(57) ABSTRACT

Provided is a method of preparing a benzenesulfonyl compound by reacting a compound represented by the following Chemical Formula,

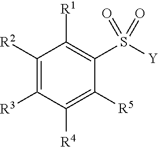

and an n-hydric alcohol in the presence of a phase transfer catalyst and an alkali metal hydroxide,
wherein
R¹ to R⁵ are independently of one another hydrogen, a halogen, C1-C7 alkoxy, or C1-C7 alkyl;
X is a residue derived from the n-hydric alcohol;
n is an integer of 1 to 4; and
Y is a halogen. The method has an excellent reaction speed even under mild reaction conditions and may produce the benzenesulfonyl compound in a high yield.

### CROSS-REFERENCE TO RELATED APPLICATIONS

This application claims priority under 35 U.S.C. § 119 to Korean Patent Application No. 10-2024-0119927, filed on Sep. 4, 2024, the disclosure of which is incorporated herein by reference in its entirety.

### TECHNICAL FIELD

The embodiments of the present disclosure relate to a method of preparing a benzenesulfonyl compound.

### BACKGROUND

A benzenesulfonyl compound is known to form a uniform SEI (solid electrolyte interface) layer on the surface of an electrode, and to enhance battery stability when used as an electrolytic solution additive. Although various structural forms of benzenesulfonyl compounds have been studied for their effectiveness as electrolyte additives, research on synthetic methods for making these compounds remains limited.

Currently, existing methods for the synthesis of a benzenesulfonyl compound have several technical difficulties which increase production costs and limit its commercial applicability. For example, it is difficult to mass produce the benzenesulfonyl compound due to the use of expensive catalysts. When less expensive, more commercially viable catalysts are used, a multi-stage process is employed with a generally low reaction rate, that significantly increases the amount of starting materials needed relative to the final product, and reduces the overall yield and productivity. Hence, a new improved method is needed.

### SUMMARY

An embodiment of the present disclosure provides a method of preparing a benzenesulfonyl compound with a substantially improved yield and productivity.

Another embodiment of the present disclosure provides a simpler, easier to use method of preparing a benzenesulfonyl compound without compromising the yield and productivity.

Accordingly, an embodiment of the present disclosure is directed to a method of preparing a benzenesulfonyl compound with a substantially improved yield and productivity and which is also easier to implement.

According to an embodiment of the present disclosure, a method of preparing a benzenesulfonyl compound is provided which includes preparing a benzenesulfonyl compound represented by the following Chemical Formula 1 by reacting a compound represented by the following Chemical Formula 2 and an n-hydric alcohol in the presence of a phase transfer catalyst and an alkali metal hydroxide aqueous solution:

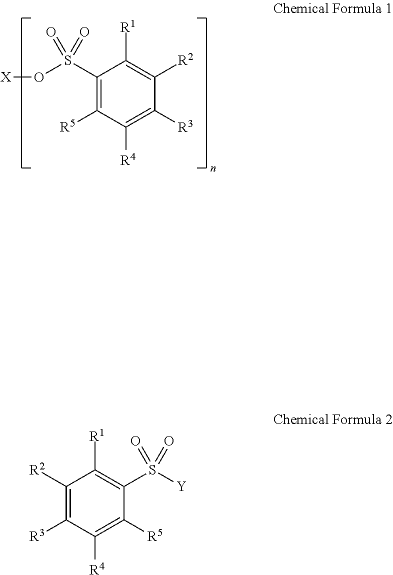

wherein, R¹ to R⁵ are independently of one another hydrogen, a halogen, C1-C7 alkoxy, or C1-C7 alkyl;
X is a residue derived from the n-hydric alcohol;
n is an integer of 1 to 4; and
Y is a halogen.

The alkali metal hydroxide aqueous solution may include 40 to 70 wt % of the alkali metal hydroxide.

The phase transfer catalyst may be an ammonium-based catalyst.

The compound represented by Chemical Formula 2 may be used at 0.3n to 1n mol with respect to 1 mol of the n-hydric alcohol, in which n may be an integer of 1 to 4. For example, in an embodiment, the n-hydric alcohol may be a tri-hydric alcohol, then n is 3 and the compound of formula 2 may be used at 0.3×3 to 1×3 i.e., 0.9 to 3 mole per 1 mole of the tri-hydric alcohol.

The compound represented by Chemical Formula 2 may be used at 0.3n to 0.8n mol with respect to 1 mol of the n-hydric alcohol, in which n may be an integer of 1 to 4.

The alkali metal hydroxide may be used at 1 mol to 5 mol with respect to 1 mol of the compound of Chemical Formula 2.

The alkali metal hydroxide and the phase transfer catalyst may be used at a mole ratio of 1:0.005 to 1:0.05.

The method of preparing the benzenesulfonyl compound according to an embodiment may be performed under a temperature condition of 20 to 40° C. for 1 to 10 hours.

In the method of preparing the benzenesulfonyl compound according to an embodiment, a yield of the benzenesulfonyl compound may be 60% or more.

Other embodiments, features, and advantages will become apparent to those with ordinary skill in the art from the following detailed description, the drawings, and the claims.

### DETAILED DESCRIPTION

In the present specification, unless otherwise defined, all technical terms and scientific terms have the same meanings as those commonly understood by a person skilled in the art. The terms used herein are only for effectively describing a certain specific embodiments and are not intended to limit the scope of the present disclosure.

A singular form used in the present specification may be intended to also include a plural form, unless otherwise indicated in context.

Throughout the present specification, unless otherwise particularly stated, the word “comprise”, “equipped”, “contain”, or “have” does not mean the exclusion of any other constituent element, but mean further inclusion of other constituent elements, and elements, materials, or processes which are not further listed are not excluded.

The numerical range used in the present specification includes all values within the range including the lower limit and the upper limit, increments logically derived from the form and spanning of a defined range, all double limited values, and all possible combinations of the upper limit and the lower limit in the numerical range defined in different forms. Unless otherwise defined in the present specification, values which may be outside a numerical range due to experimental error or rounding off of a value are also included in the defined numerical range.

Unless otherwise particularly defined in the present specification, “about” may be considered as a value within 30%, 25%, 20%, 15%, 10%, or 5% of a stated value.

Hereinafter, the embodiments of the present disclosure will be described in detail. However, it is noted that the described embodiments are provided only for illustrative purposes, and the embodiments are not limited to the specific described embodiments.

The term “halogen” in the present specification may refer to fluorine (F), chlorine (Cl), bromine (Br), or iodine (I) atom.

The term “alkyl” in the present specification is an organic radical derived from an aliphatic hydrocarbon by removal of one hydrogen and may include both straight chain and branched chain forms. The alkyl may have 1 to 7 carbon atoms, specifically 1 to 5 carbon atoms, specifically 1 to 4 carbon atoms, or specifically 1 to 3 carbon atoms. The alkyl includes, as an example, methyl, ethyl, propyl, isopropyl, butyl, isobutyl, t-butyl, pentyl, hexyl, ethylhexyl, and the like, but is not limited thereto.

The term “alkoxy” in the present specification is indicated as *—O-alkyl in which alkyl is as defined above. The alkoxy may include, for example, methoxy, ethoxy, n-propoxy, isopropoxy, n-butoxy, isobutoxy, t-butoxy, and the like, but is not limited thereto.

The term “yield” in the present specification is defined as the product of selectivity and conversion. In this context, “yield” refers to the amount of desired product obtained from a chemical reaction, expressed as a percentage of the theoretical maximum.

According to an embodiment a method of preparing a benzenesulfonyl compound is provided, which is simpler and easier to implement and at the same time provides yield and productivity improvements.

Specifically, the method of preparing a benzenesulfonyl compound according to an embodiment may include preparing a benzenesulfonyl compound represented by the following Chemical Formula 1 by reacting a compound represented by the following Chemical Formula 2 and an n-hydric alcohol in the presence of a phase transfer catalyst and an alkali metal hydroxide aqueous solution:

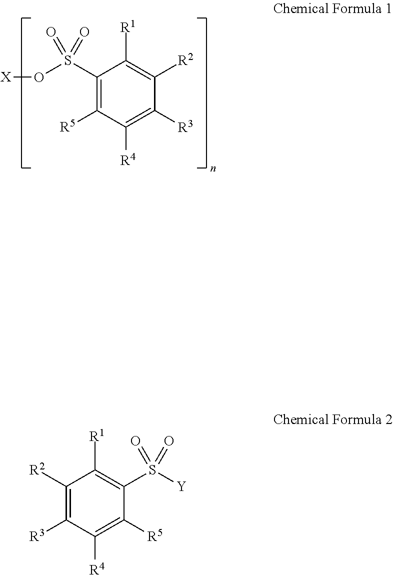

wherein
R¹ to R⁵ are independently of one another hydrogen, a halogen, C1-C7 alkoxy, or C1-C7 alkyl;
X is a residue derived from the n-hydric alcohol;
n is an integer of 1 to 4; and
Y is a halogen.

In a conventional method of preparing a benzenesulfonyl compound, when an economical base catalyst, such as an alkali metal hydroxide, is introduced for securing economic feasibility, an excessive amount of catalyst is used, and both a reaction speed and a yield are reduced. By contrast, according to the preparation method of an embodiment of the present disclosure, a combination of the phase transfer catalyst and the alkali metal hydroxide aqueous solution is used which has been found to provide an excellent reaction speed even under mild reaction conditions and may produce the benzenesulfonyl compound in a high yield.

The alkali metal hydroxide aqueous solution may include 20 to 80 wt %, 30 to 80 wt %, 40 to 80 wt %, 40 to 70 wt %, or 40 to 60 wt % of an alkali metal hydroxide and may further increase the yield. In terms of an increased product yield at an enhanced overall reaction rate even under mild reaction conditions, a content of the alkali metal hydroxide(s) included in the alkali metal hydroxide aqueous solution in a range of 30 to 70 wt % or 50 to 70 wt % may be particularly beneficial.

The alkali metal hydroxide metal may be used at 0.1 mol to 10 mol, 1 mol to 10 mol, 1 mol to 8 mol, 1 mol to 6 mol, 1 mol to 5 mol, 1 mol to 3 mol, 1.2 mol to 3 mol, 1.5 mol to 3 mol, or 1.5 mol to 2 mol with respect to 1 mol of the compound represented by Chemical Formula 2, and when the alkali metal hydroxide aqueous solution within the range of concentration described above is used to satisfy the mole ratio, the reaction speed and the yield may be improved substantially.

The alkali metal hydroxide and the phase transfer catalyst may be used at a mole ratio of 1:0.001 to 0.1, 1:0.001 to 0.05, 1:0.005 to 0.05, 1:0.005 to 0.03, or 1:0.01 to 0.03. It is noted that 1:0.001 to 0.1 means 1:0.001 to 1:0.1, 1:0.001 to 0.05 means 1:0.001 to 1:0.05 and likewise for the remaining ranges.

The alkali metal hydroxide may be sodium hydroxide (NaOH) or potassium hydroxide (KOH).

The phase transfer catalyst (PTC) may be an ammonium-based catalyst, including, for example, an ammonium halide catalyst such as tetramethylammonium bromide, tetramethylammonium chloride, tetraethylammonium bromide, tetrapropylammonium bromide, tetrabutylammonium bromide, tetrabutylammonium chloride, cetyltriammonium bromide, benzyltriethylammonium chloride, and trioctylmethylammonium chloride.

The compound represented by Chemical Formula 2 may be used at 0.2n mol or more, 0.3n mol or more and 2n mol or less, 1.5n mol or less, or In mol or less, specifically 0.2n to 2n mol, 0.3n to 1.5n mol, 0.3n to 1.2n mol, 0.3n to 1.0n mol, 0.3n to 0.9n mol, or 0.3n to 0.8n mol, with respect to 1 mol of the n-hydric alcohol, may further increase the yield of the benzenesulfonyl compound, and may include all possible combinations of the upper limit and the lower limit of the numerical range, and n may be an integer of 1 to 4 or an integer of 2 to 4.

The n-hydric alcohol refers to an alcohol having n hydroxyl groups (—OH) and may be monohydric alcohol, dihydric alcohol, trihydric alcohol, or tetrahydric alcohol. As an example, the n-hydric alcohol may be a monohydric alcohol selected from methanol, ethanol, 1-propanol, 2-propanol, 1-butanol, 2-butanol, 1-pentanol, 2-pentanol, 3-pentanol, 1-hexanol, 2-hexanol, 3-hexanol, and the like; a dihydric alcohol selected from ethylene glycol, diethylene glycol, triethylene glycol, 1,2-propanediol, 1,3-propanediol, 1,3-butanediol, 1,4-butanediol, 1,5-pentanediol, 2,2-dimethyl-1,3-propanediol, 1,6-hexanediol, and the like; a trihydric alcohol such as glycerol and trimethylolpropane; and a tetrahydric alcohol such as pentaerythritol and erythritol, but is not limited thereto.

As an example, the n-hydric alcohol may be a dihydric alcohol, and the compound represented by Chemical Formula 2 may be used at 0.5 to 3 mol, 0.5 to 2 mol, 0.6 to 2.5 mol, 0.6 to 2 mol, 0.6 to 1.8 mol, or 0.6 to 1.6 mol with respect to 1 mol of the dihydric alcohol, and may include all possible combinations of the upper limit and the lower limit of the numerical range.

As an example, the n-hydric alcohol may be a trihydric alcohol, and the compound represented by Chemical Formula 2 may be used at 0.5 to 4 mol, 0.5 to 3 mol, 1 to 3 mol, 1 to 2.5 mol, or 1 to 2 mol with respect to 1 mol of the trihydric alcohol, and may include all possible combinations of the upper limit and the lower limit of the numerical range.

As an example, R¹ to R⁵ may be independently of one another hydrogen, a halogen, C1-C4 alkoxy, or C1-C4 alkyl, and may be hydrogen, fluoro (—F), or methyl.

The preparation method according to an embodiment has an excellent reaction speed even under mild reaction conditions and may produce the benzenesulfonyl compound in a high yield. For example, the reaction may be performed under a temperature condition of 20 to 40° C. for 1 to 10 hours, 2 to 10 hours, or 3 to 8 hours, and the yield of the benzenesulfonyl compound may be 60% or more, 70% or more, 75% or more, 80% or more, or 90% or more, in which the upper limit may be 99%.

In an embodiment, the benzenesulfonyl compound obtained by the method disclosed in the present disclosure may be used as an electrolyte additive in a secondary battery or a lithium-ion battery.

Hereinafter, the embodiments described above will be described in detail through the following examples. However, the following examples are provided only for describing the embodiments of the present disclosure, and are not intended to define or limit the scope of the present disclosure.

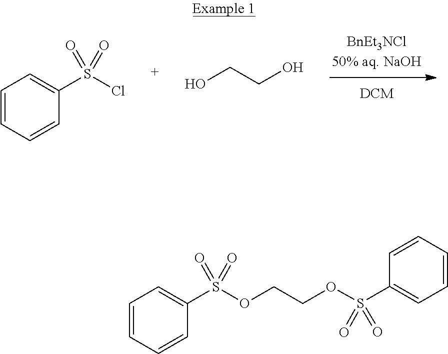

In a round bottom flask 0.15 mol of ethylene glycol, 0.005 mol of benzyltriethylammonium chloride (BnEt₃NCl), and 30 mL of dichloromethane (DCM) were added, and the flask was cooled to maintain the temperature of the mixture to about 0° C., and then 0.2 mol of benzenesulfonyl chloride was added with stirring. During the addition of the benzenesulfonyl chloride the temperature of the mixture was maintained at about 0° C. Subsequently, 0.3 mol of sodium hydroxide was added as 50 wt % of a sodium hydroxide aqueous solution. During the addition of the sodium hydroxide, the internal temperature was maintained at lower than 30° C., and after the addition, stirring was further performed at room temperature (25° C.) for 5 hours. After the reaction was finished, an organic layer was washed with water twice, concentrated, and recrystallized in methanol to obtain 1,2-bis(benzenesulfonyloxy) ethane) with 93% yield. The NMR analysis of the 1,2-bis(benzenesulfonyloxy) ethane was:

¹H-NMR (CDCl₃, 500 MHz, ppm): 7.87 (4H, m), 7.68 (2H, m), 7.56 (4H, m), 4.23 (4H, s).

### Example 2

The process was performed in the same manner as in Example 1, except that the amount of ethylene glycol used was changed to 0.13 mol.

### Example 3

The process was performed in the same manner as in Example 1, except that the amount of ethylene glycol used was changed to 0.1 mol.

### Example 4

The process was performed in the same manner as in Example 3, except that the amount of sodium hydroxide used was changed to 0.2 mol.

### Example 5

The process was performed in the same manner as in Example 3, except that 30 wt % of a sodium hydroxide aqueous solution was used instead of 50 wt % of a sodium hydroxide aqueous solution.

### Example 6

The process was performed in the same manner as in Example 1, except that the amount of benzyltriethylammonium chloride used was changed to 0.002 mol.

### Example 7

The process was performed in the same manner as in Example 1, except that the amount of ethylene glycol used was changed to 0.3 mol.

### Example 8

The process was performed in the same manner as in Example 1, except that tetrabutylammonium bromide was used instead of benzyltriethylammonium chloride.

### Example 9

The process was performed in the same manner as in Example 1, except that 50 wt % of a potassium hydroxide aqueous solution was used instead of 50 wt % of a sodium hydroxide aqueous solution.

### Comparative Example 1

The process was performed in the same manner as in Example 4, except that benzyltriethylammonium chloride was not used.

### Comparative Example 2

The process was performed in the same manner as in Example 4, except that 0.05 mol of triethylamine was used instead of benzyltriethylammonium chloride.

The mole ratio described in the following Table 1 refers to the mole ratio with respect to 1 mol of benzenesulfonyl chloride.

TABLE 1

| | Ethylene | Alkali metal hydroxide | | | | |
|---|---|---|---|---|---|---|
| | glycol | aqueous solution | | Catalyst | | Yield |
| | Mole ratio | Type | Mole ratio | Type | Mole ratio | (%) |
| Example 1 | 0.75 | 50 wt % NaOH | 1.5 | BnEt₃NCl | 0.025 | 93 |
| Example 2 | 0.65 | 50 wt % NaOH | 1.5 | BnEt₃NCl | 0.025 | 90 |
| Example 3 | 0.5 | 50 wt % NaOH | 1.5 | BnEt₃NCl | 0.025 | 81 |
| Example 4 | 0.5 | 50 wt % NaOH | 1.0 | BnEt₃NCl | 0.025 | 73 |
| Example 5 | 0.5 | 30 wt % NaOH | 1.5 | BnEt₃NCl | 0.025 | 65 |
| Example 6 | 0.75 | 50 wt % NaOH | 1.5 | BnEt₃NCl | 0.01 | 88 |
| Example 7 | 1.5 | 50 wt % NaOH | 1.5 | BnEt₃NCl | 0.025 | 95 |
| Example 8 | 0.75 | 50 wt % NaOH | 1.5 | Bu₄NBr | 0.025 | 91 |
| Example 9 | 0.75 | 50 wt % KOH | 1.5 | BnEt₃NCl | 0.025 | 92 |
| Comparative | 0.5 | 50 wt % NaOH | 1.0 | — | — | 56 |
| Example 1 | | | | | | |
| Comparative | 0.5 | 50 wt % NaOH | 1.0 | Et3N | 0.25 | 59 |
| Example 2 | | | | | | |

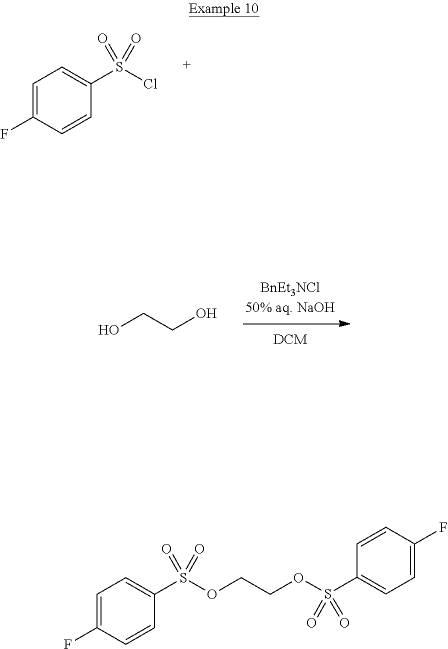

1,2-bis(4-fluorobenzenesulfonyloxy)ethane)) was obtained (with a yield of 94%) in the same manner as in Example 1, except that 4-fluorobenzenesulfonyl chloride was used instead of benzenesulfonyl chloride. The NMR analysis was:

¹H-NMR (CDCl₃, 500 MHz, ppm): 7.89 (4H, m), 7.23 (4H, m), 4.25 (4H, s).

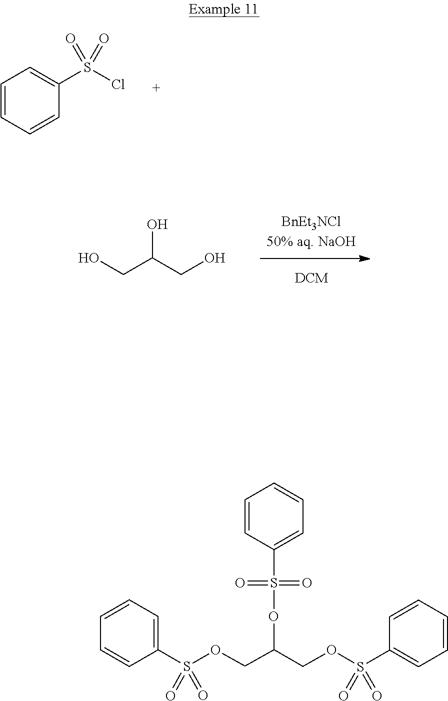 [SMILES: O=S(=O)(Cl)c1ccccc1 ; OCC(O)CO ; O=S(=O)(OCC(COS(=O)(=O)c1ccccc1)OS(=O)(=O)c1ccccc1)c1ccccc1]

1,2,3-tris(benzenesulfonyloxy)propane)) was obtained (with a yield of 90%) in the same manner as in Example 1, except that 0.1 mol of glycerol was used instead of 0.15 mol of ethylene glycol. The NMR analysis was:

¹H-NMR (CDCl₃, 500 MHz, ppm): 7.82 (6H, m), 7.68 (3H, m), 7.55 (6H, m), 4.74 (1H, p), 4.13 (4H, d).

The method of preparing a benzenesulfonyl compound according to embodiments of the present disclosure provides a benzenesulfonyl compound in an excellent yield by a simple, easy to use process. Specifically, the preparation method has an excellent reaction speed even under mild reaction conditions to have a short reaction time, and can produce a benzenesulfonyl compound in a high yield by using economical materials. The method further exhibits excellent productivity and may be advantageous for many industrial applications.

Hereinabove, although the embodiments of the present disclosure have been described by reference to specific embodiments, it is understood that these embodiments have been provided only for assisting in the entire understanding of the present disclosure, and the embodiments of the present disclosure are not limited to the specific embodiments. Various other embodiments, modifications, and changes may be made by those skilled in the art to which the present disclosure pertains from the description without departing from the scope of the present disclosure as defined in the appended claims. Furthermore, the embodiments may be combined to form additional embodiments.

What is claimed is:

1. A method of preparing a benzenesulfonyl compound, the method comprising:
preparing a benzenesulfonyl compound represented by the following Chemical Formula 1 by reacting a compound represented by the following Chemical Formula 2 and an n-hydric alcohol in the presence of a phase transfer catalyst and an alkali metal hydroxide aqueous solution: 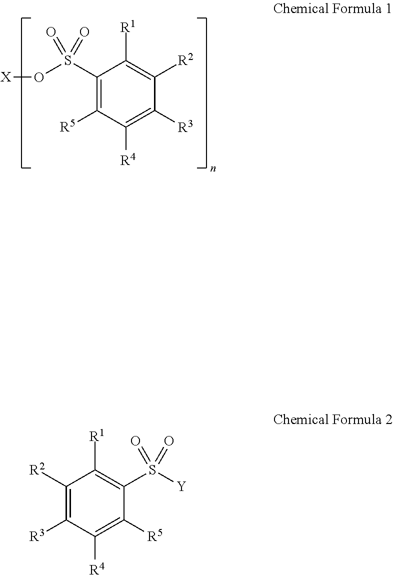
wherein
R¹ to R⁵ are independently of one another hydrogen, a halogen, C1-C7 alkoxy, or C1-C7 alkyl;
X is a residue derived from the n-hydric alcohol;
n is an integer of 1 to 4; and
Y is a halogen.

2. The method of preparing a benzenesulfonyl compound of claim 1, wherein the alkali metal hydroxide aqueous solution includes 40 to 70 wt % of the alkali metal hydroxide.

3. The method of preparing a benzenesulfonyl compound of claim 1, wherein the phase transfer catalyst is an ammonium-based catalyst.

4. The method of preparing a benzenesulfonyl compound of claim 1, wherein the compound represented by Chemical Formula 2 is used at 0.3n to in mol with respect to 1 mol of the n-hydric alcohol, in which n is an integer of 1 to 4.

5. The method of preparing a benzenesulfonyl compound of claim 4, wherein the compound represented by Chemical Formula 2 is used at 0.3n to 0.8n mol with respect to 1 mol of the n-hydric alcohol, in which n is an integer of 1 to 4.

6. The method of preparing a benzenesulfonyl compound of claim 1, wherein the alkali metal hydroxide is used at 1 mol to 5 mol with respect to 1 mol of the compound of Chemical Formula 2.

7. The method of preparing a benzenesulfonyl compound of claim 6, wherein the alkali metal hydroxide and the phase transfer catalyst are used at a mole ratio of 1:0.005 to 1:0.05.

8. The method of preparing a benzenesulfonyl compound of claim 1, wherein the reaction is performed under a temperature condition of 20 to 40° C. for 1 to 10 hours.

9. The method of preparing a benzenesulfonyl compound of claim 1, wherein a yield of the benzenesulfonyl compound is 60% or more.

10. A method of preparing a benzenesulfonyl compound, the method comprising:
preparing a benzenesulfonyl compound represented by the following Chemical Formula 1 by reacting a compound represented by the following Chemical Formula 2 and an n-hydric alcohol in the presence of a phase transfer catalyst and an alkali metal hydroxide aqueous solution: 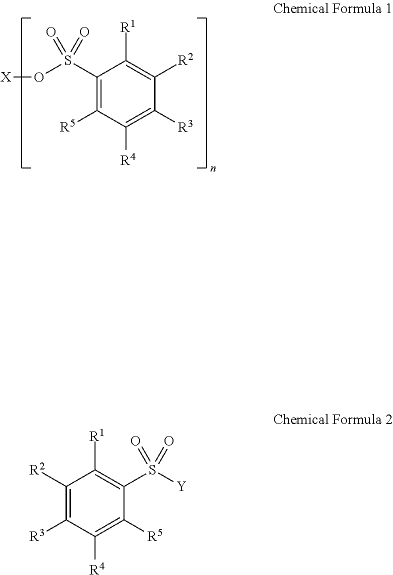
wherein
R¹ to R⁵ are independently of one another hydrogen, a halogen, C1-C7 alkoxy, or C1-C7 alkyl;
X is a residue derived from the n-hydric alcohol;
n is an integer of 1 to 4; and
Y is a halogen,
wherein the alkali metal hydroxide is used at 1 mol to 5 mol with respect to 1 mol of the compound of Chemical Formula 2, wherein the alkali metal hydroxide and the phase transfer catalyst are used at a mole ratio of 1:0.005 to 0.05, and
wherein the reaction is performed under a temperature condition of 20 to 40° C. for 1 to 10 hours.

11. The method of preparing a benzenesulfonyl compound of claim 10, wherein a yield of the benzenesulfonyl compound is 90 or more.

12. The method of preparing a benzenesulfonyl compound of claim 11, wherein the benzenesulfonyl compound is:
1,2,3-tris(benzenesulfonyloxy)propane), or
1,2-bis(4-fluorobenzenesulfonyloxy)ethane)).
